(19) United States
(12) Patent Application Publication
(10) Pub. No.: US 2023/0329568 A1
(43) Pub. Date: Oct. 19, 2023

(54) Wearable Blood Pressure Monitor

(71) Applicant: Stanley Lawrence, Covington, GA (US)

(72) Inventor: Stanley Lawrence, Covington, GA (US)

(21) Appl. No.: 17/722,492

(22) Filed: Apr. 18, 2022

(51) Int. Cl. A61B 5/022; A61B 5/021; A61B 5/00; A61B 5/0205; G04G 21/02

(52) U.S. Cl. CPC A61B 5/02233; A61B 5/02141; A61B 5/681; A61B 5/7425; A61B 5/0008; A61B 5/7475; A61B 5/02055; G04G 21/025

(57) ABSTRACT

A blood pressure monitoring watch includes a wristband and a housing unit. The wristband is disposed on opposing sides of the housing unit. The wristband includes a closure fastener and a corresponding fastener receiver. The housing unit further includes an electronic display screen, a sensor, a processor, a memory, and a wireless transceiver. The wristband includes a plurality of inflatable bladders. The plurality of inflatable bladders is operably connected to an internal pump, whereby, the plurality of inflatable bladders apply pressure to a wearer's wrist to measure the blood pressure. The sensor is configured to detect the blood pressure of the wearer when the wearer inflates the plurality of inflatable bladders. The display screen is configured to display blood pressure readings and other information, and the wireless transceiver is configured to communicate the information with an external device.

### CROSS REFERENCE TO RELATED APPLICATIONS

This application claims the benefit of U.S. Provisional Application No. 63/123,420 filed on Dec. 9, 2020. The above identified patent application is herein incorporated by reference in its entirety to provide continuity of disclosure.

### BACKGROUND OF THE INVENTION

The present invention relates to a wearable blood pressure monitor. More specifically, the present invention provides a wearable blood pressure monitor that includes a plurality of inflatable air bladders and a wireless transceiver for communicating information between external devices.

Monitoring blood pressure is a proactive way to prevent an individual from developing any further health complications. With varying levels of blood pressure, individuals may be unaware of an undesired rise or fall of their blood pressure as they go throughout a day. An individual may only measure their blood pressure at schedule times during the day. However, the time intervals between the schedule measurements may be when their blood pressure rises or drops to dangerous levels. Untreated high blood pressure can lead to other serious conditions like a stroke or heart attack. Monitoring the blood pressure throughout different times of the day with ease will assist the individual controlling their blood pressure levels and prevent any undesired results from occurring.

There are an abundance of people who do not perform routine blood pressure checks on themselves. Typically, the lack of convenience of the at-home blood pressure monitors is a contributing factor to the limited ability of individuals who want to regularly check their blood pressure throughout the day. The currently available at-home blood pressure monitors are bulky and cumbersome to wear, continuously. Often those types of blood pressure monitors are designed to be worn only for the duration of the blood pressure test. After which, the user will remove the blood pressure monitor off their arm as soon as a blood pressure reading is provided. This will leave considerable periods of time during the day which go unmonitored because the blood pressure monitors are inaccessible for use. The lapse of time between testing blood pressure may lead to a potential health complication that might have been avoidable if properly monitored.

There have been a recent move to the use of continuous blood pressure monitor which are accessible for a wearer. These current methods of continuous blood pressure monitoring do not measure the blood pressure the same way as the inflatable cuffs. The inflatable cuffs inflate an air bladder that applies pressure to the cardiovascular system of the user, like the procedure of a health professional, that allows for a monitor to display the systolic and diastolic blood pressure. The current methods of continuous blood pressure monitoring instead use sensors that attempt to give a reading to the user. Although these sensors provide a close approximation of the actual blood pressure, they do not provide as accurate of a reading as applying pressure to the cardiovascular system with an air bladder.

Therefore, there is a defined need amongst the known prior art references for a modified digital smartwatch capable of measuring the wearer's blood pressure with a plurality of manually inflatable air bladders and a wireless transceiver for communicating sensed information between external devices.

### SUMMARY OF THE INVENTION

In view of the foregoing disadvantages inherent in the known types of blood pressure monitors now present in the known art, the present invention provides a new blood pressure monitor wherein a plurality of inflatable air bladders and a wireless transceiver for communicating sensed information between external devices.

The present device comprises a plurality of inflatable air bladders. The plurality of inflatable air bladders is disposed on the interior surface of the device. So that, the plurality of inflatable air bladders will be positioned against the skin of the wearer. When the wearer chooses to engage the device, air will inflate the plurality of inflatable air bladders as to apply pressure to the cardiovascular system located within the wearer's wrist. The pressure that is generated from the plurality of inflatable air bladders will disrupt the flow of blood within the cardiovascular system to generate a reading of the wearer's systolic and diastolic blood pressure. After the detection of both a systolic and a diastolic blood pressure, the plurality of inflatable air bladders will deflate to restore the flow of blood within the cardiovascular system. The present device permits the biometric readings to be displayed on an electronic display screen or to be transmitted to a connected electronic device.

Other objects, features, and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a top view of an embodiment of the blood pressure monitoring device.

FIG. 1B shows a top view of an alternative embodiment of the blood pressure monitoring device.

FIG. 2A shows a side view of an alternative embodiment of the blood pressure monitoring device.

FIG. 2B shows a perspective view of an alternative embodiment of the blood pressure monitoring device.

FIG. 3 shows a close-up view of an embodiment of the blood pressure monitoring device.

### DETAILED DESCRIPTION OF THE INVENTION

Reference is made herein to the attached drawings. Like reference numerals are used throughout the drawings to depict like or similar elements of blood pressure monitoring device. The figures are intended for representative purposes only and should not be limiting in any respect.

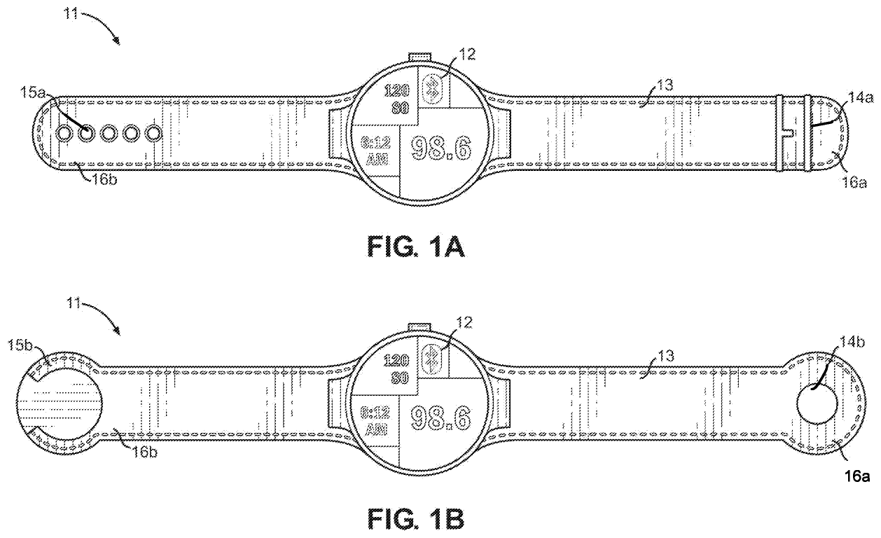

FIG. 1A shows a top view of an embodiment of a blood pressure monitoring device. The blood pressure monitoring device 11 comprises an electronic display screen 12 and a wristband 13. The wristband 13 includes an external side and an internal side. The wristband 13 comprises a pair of opposing ends. The pair of opposing ends of the wristband 13 are on opposing sides of the electronic display screen 12. The electronic display screen 12 is centrally located between the pair of opposing ends of the wristband 13.

A first end 16a of the pair of opposing ends includes a closure fastener 14a. A second end 16b of the pair of opposing ends includes a fastener receiver 15a, whereby the fastener receiver 15a corresponds with the closure fastener 14a. In one embodiment, the closure fastener 14a is a buckle. In the same embodiment, the corresponding fastener receiver 15a is a plurality of apertures. In said embodiment, the plurality of apertures is disposed along the wristband 13 from the second end 16b towards the electronic display screen 12. In use, the closure fastener 14a will engage with the corresponding fastener receiver 15a to secure the first end 16a of the wristband 13 with the second end 1b wristband 13 about the wrist of the user.

FIG. 1B shows a top view of an alternative embodiment of a blood pressure monitoring device. The blood pressure monitoring device 11 comprises an electronic display screen 12 and a wristband 13. The wristband 13 includes an external side and an internal side. The wristband 13 comprises a pair of opposing ends. The pair of opposing ends of the wristband 13 are on opposing sides of the electronic display screen 12. A first end 16a of the pair of opposing ends includes a closure fastener 14b. A second end 16b of the pair of opposing ends includes a corresponding fastener receiver 15b. In one embodiment, the closure fastener 14b is a rounded head. In the same embodiment, the corresponding fastener receiver 15b is a rounded inlet. In use, the closure fastener 14b will engage with the corresponding fastener receiver 15b to secure the first end 16a of the wristband 13 with the second end 1b wristband 13 about the wrist of the user.

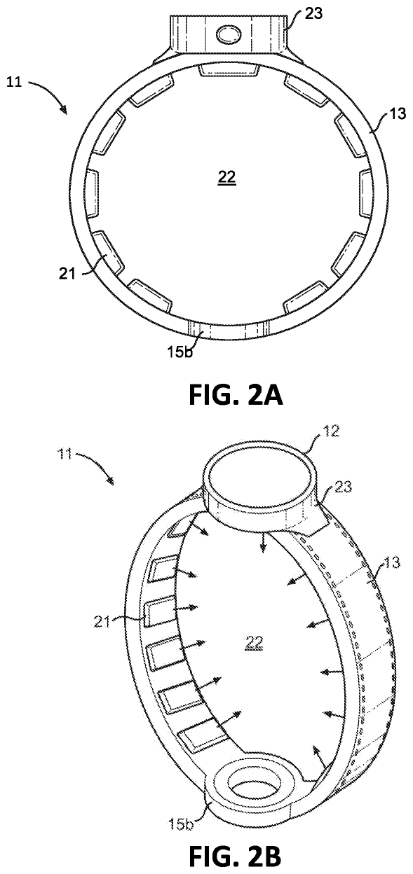

FIG. 2A shows a side view of an alternative embodiment of a blood pressure monitoring device. The blood pressure monitoring device 11 further comprises a plurality of inflatable bladders 21. The plurality of inflatable bladders 21 is disposed on the internal side of the wristband 13. When the corresponding fastener receiver 15b engages with the closure fastener, the wristband 13 is enclosed. When the wristband 13 is enclosed an internal space 22 is defined. When the blood pressure monitoring device 11 is in use, the wristband 13 will encircle the wearer's wrist, wherein the wearer's wrist is positioned within the internal space 22. In that configuration, plurality of inflatable bladders 21 will be in direct contact of the user's wrist.

When the corresponding fastener receiver 15b is engaged with a closure fastener the wristband 13 will be a closed loop. The closed loop wristband 13 will create an internal space 22. In use, the lower part of the user's arm will be within the internal space 22 whereby the blood pressure monitoring device 11 encircles the user's wrist. The electronic display screen is secured on a housing unit 23. The housing unit 23 is secured to the wristband 13. When the blood pressure monitoring device 11 was secured to the user's wrist, the housing unit 23 is positioned on the top of the user's wrist. A plurality of inflatable bladders 21 is disposed on the internal side of the wristband 13. An actuator disposed on the housing unit 23 is operably connected to the plurality of inflatable bladders 21. Additionally, the blood pressure monitoring device 11 includes an infrared sensor. The infrared sensor is disposed on the internal side of the wristband 13. When selected by the user, the infrared sensor will measure the user's internal body temperature.

FIG. 2B shows a perspective view of an alternative embodiment of a blood pressure monitoring device. When the actuator is engaged on the housing unit 23, an internal pump will activate. The plurality of inflatable bladders 21 is operably connected to the internal pump. When the internal pump is activated, the internal pump will fill the plurality of inflatable bladders 21 with air. As the plurality of inflatable bladders 21 are inflated with air, the plurality of inflatable bladders 21 will apply a pressure to prevent blood flow through the user's arm. Specifically, the pressure from the inflated plurality of inflatable bladders 21 will block the blood flow in the user's Brachial artery. While the blood flow in the Brachial artery is occulated, a sensor will measure a systolic blood pressure of the user. When the systolic blood pressure is measured, the plurality of inflatable bladders 21 will rapidly deflate and the pressure is then gradually released. At that time, the blood flow in the brachial artery is no longer occulated and will continue through the user's cardiovascular system. The instant the blood flow is no longer occulated, the sensor will measure the diastolic blood pressure. The systolic blood pressure and the diastolic blood pressure will be displayed on the electronic display screen 12.

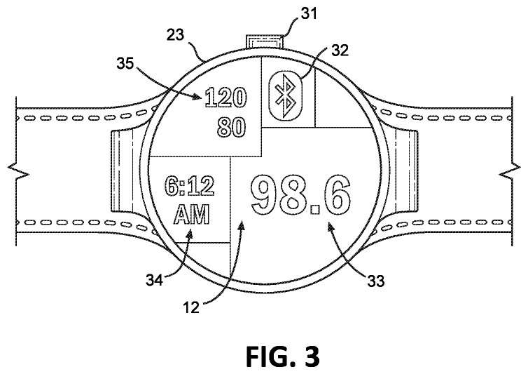

FIG. 3 shows a close-up view of an embodiment of the blood pressure monitoring device. The blood pressure monitoring device includes the housing unit 23. The wristband is disposed on opposing ends of the housing unit 23. In the shown embodiment, the housing unit 23 is rounded. The housing unit 23 comprises a top face, a bottom face, and a surrounding sidewall. The surrounding sidewall is sandwiched between the top face and the front face. The housing unit 23 further comprises an electronic display screen 12 and an actuator 31. The electronic display screen 12 is disposed on the top face of the housing unit 23. The actuator 31 is disposed on the surrounding sidewall of the housing unit 23. In one embodiment, the actuator 31 is a push button. The actuator 31 is operably connected to the electronic display screen 12. The actuator 31 permits the user to select the information they want to display on the electronic display screen 12.

The electronic display screen 12 is configured to display a plurality of biometrics of the wear. A sensor is disposed on the bottom face of the housing unit 23. The sensor is operably connected to the actuator 31 and the electronic display screen 12. A first biometric of the plurality of biometrics shown on the electronic display screen 12 is blood pressure 35. The sensor reads the diastolic and systolic blood pressure of the wearer when the plurality of inflatable bladders 21 oculate the blood flow in the brachial artery. The sensor then sends the readings to the electronic display screen 12. The blood pressure 35 displays both the diastolic and systolic blood pressures of the wearer. A second biometric of the plurality of biometrics shown on the electronic display screen 12 is body temperature 33. A temperature sensor is disposed on the bottom face of the housing unit 23. The temperature sensor reads the body temperature of the wearer. The temperature sensor then sends the readings to the electronic display screen 12. The body temperature 33 may display in both the Celsius and Fahrenheit temperature.

Additionally, the electronic display screen 12 will display a clock 34. The clock 34 will display the time of day. The blood pressure monitoring device may be coupled to an electronic device. A user will link the desired electronic device to the blood pressure monitoring device via a transceiver 32. In the shown embodiment, the transceiver 32 is connected to the electronic device through a wireless Bluetooth connection. The transceiver 32 can transmit the biometric of the plurality of biometrics measured by the blood pressure monitoring device to an electronic device.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A blood pressure monitoring device, comprising:
a wristband;
wherein the wristband includes an external side and an internal side;
the wristband further comprising a pair of opposing ends and a housing unit;
whereby the housing unit includes a top face and a bottom face;
the housing unit further comprises a multifunctional sensor, a processor, a memory, and a wireless transceiver;
wherein the wireless transceiver connects to an external device;
the top face of the housing unit further includes an electronic display screen
the electronic display screen exhibits a plurality of biometrics;
a closure fastener disposed on a first end of the pair of opposing end;
a corresponding fastener receiver disposed on a second end of the pair of the opposing ends;
a plurality of inflatable bladders disposed on the internal side of the wristband;
an actuator disposed on the housing unit;
wherein the actuator is operably connected to the plurality of inflatable bladders; and
whereby engaging the actuator inflates the plurality of inflatable bladders via an internal pump.

2. The blood pressure monitoring device of claim 1, wherein the wristband encircles the wrist of a user; whereby the internal side is in direct contact with the user when the closure fastener engages with the corresponding fastener receiver.

3. The blood pressure monitoring device of claim 1, wherein the closure fastener is a buckle and the corresponding fastener receiver is a plurality of apertures.

4. The blood pressure monitoring device of claim 1, wherein the closure fastener is a rounded head and the corresponding fastener receiver is a rounded inlet.

5. The blood pressure monitoring device of claim 1, wherein the actuator is a push button.

6. The blood pressure monitoring device of claim 1, wherein the multifunctional sensor measures the body temperature of a user and utilizes the plurality of inflatable bladders to determine the blood pressure of a user.

7. The blood pressure monitoring device of claim 1, wherein the plurality of biometrics shown on the electronic display screen includes blood pressure and body temperature.

8. The blood pressure monitoring device of claim 1, wherein the electronic display screen further exhibits the time of day.

9. The blood pressure monitoring device of claim 1, wherein the wireless transceiver is configured to transmit the plurality of biometrics to the external device.
